(19) United States
(12) Patent Application Publication
(10) Pub. No.: US 2011/0172763 A1
(43) Pub. Date: Jul. 14, 2011

(54) Matrix Coated Stent

(76) Inventor: Robert Ndondo-Lay, Temecula, CA (US)

(21) Appl. No.: 13/120,011

(22) PCT Filed: Sep. 29, 2009
(86) PCT No.: PCT/US09/58900

§ 371 Date: Mar. 21, 2011

Related U.S. Application Data
(60) Provisional application No. 61/194,711, filed on Sep. 29, 2008.

(51) Int. Cl. A61F 2/01; B05D 7/00

(52) U.S. Cl. 623/1.42; 427/2.25

(57) ABSTRACT

The present invention relates generally to a drug eluting stent containing metallic surfaces modified in microsphere metallic matrix structure and methods for making same. More specifically, the invention relates to an expandable and implantable vascular stent having at least one matrix layer that promotes improved cellular adhesion properties for healing promotion healing and long term biocompatibility. In the case of coronary stents, the metallic matrix layer promotes re-endothelialization at sites of stent implantation, improves overall healing, and reduces inflammation and intimal disease progression. The microsphere metallic matrix layer may be optionally loaded with one or more therapeutic agent to further improve the function of the implanted stent and further augment clinical efficacy and safety. The active compounds are selected primarily for their anti-proliferative, immunosuppressive, and anti-inflammatory activities, among other properties, which prevent, in part, smooth muscle cell proliferation and promote endothelial cell growth.

### CROSS-REFERENCE TO RELATED APPLICATION

U.S. provisional application No. 61/194,711 dated Sep. 29, 2008 the contents of which are hereby incorporated by reference.

### BACKGROUND OF THE INVENTION

1. Field of the Invention

The present invention relates to stents, and in particular to vascular, billiary, and neural stents that may have improved efficacy and safety via surface treatment that promotes endothelialization with or without pharmaceutical applications. The stent may also contain drug or other biological agents for treatment of arthrosclerosis disease or other vessel inflammation such formation of thrombosis.

2. Description of the Prior Art

The stent procedure is fairly common, and various types of stents have been developed and used. Several types of these endoprostheses are known, including balloon expandable, self-expanding, and endoprostheses constructed from biostable springs or tubes. Different types of stent, including vascular grafts and graft-stent combinations can be provided with bio-active agents and used for minimally invasive procedures in body conduits.

Stents are used not only as a mechanical intervention of vascular conditions but also as a vehicle for providing biological therapy. As a mechanical intervention, stents act as scaffoldings, functioning to physically hold open and, if desired, to expand the wall of the passageway. Typically, stents are capable of being compressed, so that they can be inserted through small vessels via catheters, and then expanded to a larger diameter once they are at the desired location. Examples in patent literature disclosing stents which have been applied in Percutaneous Transluminal Coronary Angioplasty (PTCA) procedures include stents illustrated in U.S. Pat. No. 4,733,665 issued to Palmaz, U.S. Pat. No. 4,800,882 issued to Gianturco, and U.S. Pat. No. 4,886,062 issued to Wiktor. This and all other referenced patents are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

However, the placement of a stent in a blood vessel may injure the vessel and cause lesions in the walls of the vessel. Mechanical injury induced by stent implantation can cause endothelial denudation, which is directly associated with the formation of lesions in the vessel wall. The formation of lesions in the blood vessel wall can initiate an inflammatory response within the vasculature wall of a blood vessel. As such, this can cause the activation of circulating platelets, the infiltration of neutrophils and monocytes, and the release of pro-inflammatory cytokines and growth factors. Inflammation is a major stimulus for alteration of smooth muscle cell phenotype, and can result in smooth muscle cell activation, proliferation, and migration into the neointima, which causes restenosis. Also, recent studies suggest that such alterations in smooth muscle cell phenotype may be a result of smooth muscle cell differentiation into a myofibroblast phenotype. Thus, the physiological response to the mechanical injury caused by a stent can induce restenosis.

Additionally, mechanical injury induced by stent implantation may also cause proliferation and migration of vascular endothelial cells. The proliferation and migration of vascular endothelial cells can induce the re-endothelialization of the stented blood vessel so as to reduce lesion thrombosis. In instances that lesions in vessel wall are not re-endothelialized, lesion thrombosis can occur, which is problematic. As such, there is a need to reduce restenosis and thrombosis after stent implantation.

To reduce the possibility of restenosis and to locally deliver a biologically active material to a patient's lumen, various types of biologically active material-coated stents have been proposed. For example, U.S. Pat. No. 6,258,121 to Yang et al. discloses a stent having a polymeric coating for controllably releasing an included active agent such as paclitaxel, to inhibit restenosis following angioplasty.

Biological therapy can be achieved by medicating the stents. These are called Drug Eluting Stents (DES). DES provide for the local administration of a therapeutic substance at the diseased site. Local delivery is a preferred method of treatment in that smaller total levels of medication are administered in comparison to systemic dosages, but are concentrated at a specific site. Local delivery thus produces fewer side effects and achieves more favorable results.

The introduction of DES into clinical cardiology at the beginning of the new millennium can be considered a success story opening the gates for a new era in interventional cardiology. Comprehensive understanding of the molecular and cellular basis of neointimal hyperplasia, which ultimately accounts for in-stent restenosis, has enabled the identification of compounds that efficiently inhibit mitogen-induced smooth muscle cell proliferation, the leading cause of in-stent neointimal hyperplasia and consequently restenosis. Currently, four U.S. Food and Drug Administration-approved DES platforms are commercially available: Taxus (Boston Scientific, Boston, Mass.), Cypher® (Cordis, Johnson and Johnson, Miami Lakes, Fla.), Endeavor (Medtronic, Santa Rosa, Calif.) and Xience (Abbot Vascular, Santa Clara, Calif.). Various studies have shown that these DES efficiently prevent angiographic and clinical restenosis rates compared with bare-metal stents. The compounds applied on these particular DES platforms are different: Cypher elutes sirolimus (SRL), Taxus releases paclitaxel (PTX), Endeavor elutes ABT 578 while Xience releases everolimus. It should be noted that the two ABT 578 and everolimus are sirolimus analog. Sirolimus is also commercially known as rapamycin.

Further, although DES coronary artery stents have shown superior short- and mid-term results in lower rates of neovascularization compared to bare metal (BM) stents, long term (≧2 years) restenosis rates over 5-15% at 3 year post-procedure are still considerable due to “late thrombosis,” and are not significantly better than BM stents in certain patient groups. Moreover, in diabetic patients, restenosis rates of DES are as high as 20-30%, and these rates are even higher for BM stents for this group.

Polymeric materials, for example, are commonly used in DES as matrices for the retention of therapeutic agents. These polymeric materials are typically applied as coatings to the stent, raising issues regarding coating adhesion, mechanical properties, cracking, delamination, and material biocompatibility. Additionally problems occur when mechanical forces are applied on a stent during manufacture (e.g., crimping, stenting retention procedures, packaging etc.) as well as during actual use (e.g., unsheathing, catheter preparation, advancement through catheter and vasculature), which may result in damaging the polymeric coating. Also, many polymers with desirable controlled release properties, like the family of biodegradable polymers based on polylactide, polyglycolide and their copolymers are difficult candidates for a polymeric endoprosthetic coating, because of poor adhesion properties to metals and/or poor elongation and brittle characteristics.

It has been found that rapamycin-coated stents decrease the risk of stent-induced restenosis by inhibiting the proliferative response associated with endothelial denudation. While DES, such as stents loaded with rapamycin, may provide favorable responses to inhibit restenosis, the drugs and or polymers eluted from the stent may also lead to thrombosis. In part, this may be because the drug/polymer combination inhibits endothelial cell migration, which in turn inhibits the re-endothelialization of lesions, thereby leading to thrombosis. As such, there is a need for a drug-eluting stent that does not inhibit the re-endothelialization of lesions that are caused from implantation of the stent. Thus, there is a need for a DES that is balanced between inhibiting restenosis while permitting re-endothelialization of lesions, and thereby inhibiting thrombosis.

Therefore, it would be advantageous to have a stent and method of use thereof that inhibits restenosis and thrombosis. Also, it would be advantageous to have a stent and method of use thereof that inhibits phosphorylation, and thereby inhibits cell proliferation, but allows for endothelial cell migration to re-endothelialize lesions in the vessel wall so as to inhibit thrombosis at earlier or later stage.

There exists a need in the art for DES capable of retaining a therapeutic agent in the stent so that the drug may be eluted to a local region of the vessel wall in a controlled manner through matrix of the surface without the usage of polymer. Furthermore, an ideal drug eluting stent would encapsulate agents within the matrix in protective embodiment to reduce or eliminate current aggressive manufacturing method problems or actual use to prevent physical damage of the stent. Yet to eliminate polymer, the structure must be modified to create a field to allow the cell to growth. Several techniques have been proposed in the ongoing effort to adhere an appropriate layer to substrates, for example Hahn proposed in a U.S. Pat. No. 3,605,123 that a dense base metal be coated with a porous film of the same material. Also, U.S. Pat. Nos. 3,855,638, 4,038,713, 4,101,984, and 4,524,539 also use single size metallic particles to create a porous coat using flame plasma process.

Additional stent designs suitable DES include, for example, vascular stents such as self-expanding stents and balloon expandable stents. Examples of self-expanding stents illustrated in U.S. Pat. Nos. 4,655,771, 5,061,275 and 4,954,126 issued to Wallsten. Examples of appropriate balloon-expandable stents are shown in U.S. Pat. No. 5,449,373 issued to Pinchasik.

Although various stents are known to the art, all, or almost all of them suffer from one or more than one disadvantage. Therefore, there is a need to provide improved drug eluting stent matrix.

### SUMMARY OF THE INVENTION

It is an object of the present invention to provide a means for overcoming the difficulties associated with the methods and devices currently in use, as briefly described above. In addition, the methods for maintaining the matrix structure and the modifications ensure that the active agents reach the target site. It is desirable to modify the surface of the coronary stents, in order to confer on these devices the ability to carry and elute therapeutic agents and promote endothelialization of the vessel lining membrane.

Another embodiment of the present invention relates to metallic surface modification as an alternative means of enabling targeted delivery of therapeutic agents from medical devices. The said surface modification results in one or more layers of microspherical structure of honeycomb like metallic matrix on the surface of the stent. The matrix is loaded with the therapeutic agent of choice, or a combination of such agents.

Another embodiment of the present invention is directed toward producing a strongly adherent and mechanically robust metallic matrix, while simplifying device manufacture and loading of therapeutic agents. The metallic matrix is generated by the process of microspheric metallic sintering.

Another embodiment of the present invention also comprises unique loading methods which, independently or in conjunction with the ability to vary morphology, allow one or more therapeutic agents to be loaded into the matrix to achieve desired elution profiles.

It is to be understood that the foregoing general description and the following detailed description are exemplary and explanatory only and are not to be viewed as being restrictive of the present invention, as claimed. Further advantages of this invention will be apparent after a review of the following detailed description of the disclosed embodiments which are illustrated schematically in the accompanying drawings and in the appended claims.

### BRIEF DESCRIPTION OF THE FIGURES

In the following, embodiments of the present invention will be explained in detail on the basis of the drawings, in which:

FIG. 1 is an isometric and an enlarged view of the stent cross-section according to an embodiment of the invention.

FIG. 2A is a scanning electronic microscopic view of a DES with expanded struts according to the embodiment of the invention.

FIG. 2B is a magnified view of scanning electronic microscopic view if FIG. 2A.

FIG. 2C is a magnified view of scanning electronic microscopic view of the spherical matrix on the DES according to an embodiment of the invention.

FIG. 3A is a scanning electronic microscopic view of a DES in accordance with one embodiment of the invention.

FIG. 3B is a magnified view of FIG. 3A according to the embodiment of the invention.

FIG. 3C is an isometric and an enlarged view of a stent according to another embodiment of the invention.

FIG. 3D is a perspective drawing, side and cross sectional views of an idealized stent strut.

FIG. 3E is a perspective view of a stent strut surface for purpose of calculation according to a preferred embodiment of the invention.

FIG. 4A is a scanning electronic microscopic view of the polymer delamination, Prior art.

FIG. 4B is a scanning electronic microscopic view of the polymer delamination, Prior art.

FIG. 5 is a scanning electronic microscopic view of histological study of Endothelial lining in 7 days rabbit of the Drug Eluting Stent, Prior art.

### DETAILED DESCRIPTION

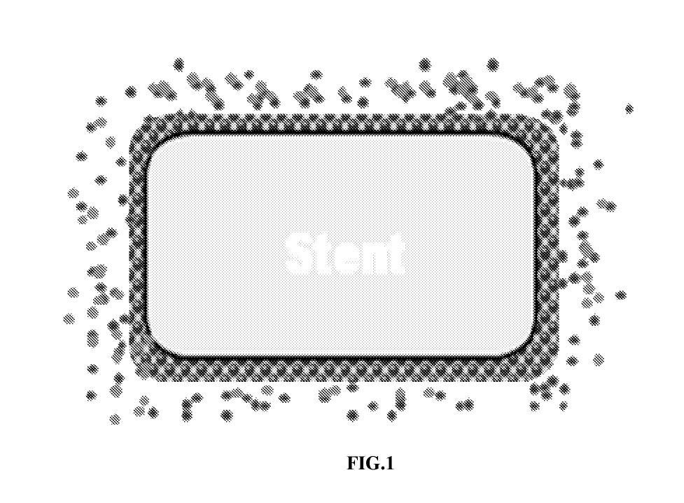

FIG. 1 illustrates a preferred embodiment where the stent strut is coated on its entire exposed surface with a matrix. The matrix is typically between 50 and 150 micron thick. The matrix can be similar to or different than the underlying stent and can be manufactured from any of the following: cobalt-chromium alloys (e.g., ELGILOY), stainless steel (316L), “MP35N,” “MP20N,” ELASTINITE (Nitinol), tantalum, tantalum-based alloys, nickel-titanium alloy, platinum, platinum-based alloys such as, e.g., platinum-iridium alloy, iridium, gold, magnesium, titanium, titanium-based alloys, zirconium-based alloys, or combinations thereof. “MP35N” and “MP20N” are trade names for alloys of cobalt, nickel, chromium and molybdenum available from Standard Press Steel Co. of Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum.

An alternative embodiment could have a matrix coated on any combination of sides of the stent strut. For example, the abluminal surface and the side surfaces would be most likely to be in direct contact with the lesion and tissue of the vessel, while the luminal side of the stent would be exposed to the blood stream. This design would potentially reduce the total amount of drug to be incorporated into the matrix.

Stents suitable for the present invention include, but are not limited to, those that have a tubular or cylindrical like portion. For example, the tubular portion of the medical device need not be completely cylindrical. The cross section of the tubular portion can be any shape, such as rectangle, a triangle, etc., not just circular. Suitable substrate of the stents of the present invention may be fabricated from a metallic material, ceramic material, polymeric or non-polymeric material, or a combination thereof preferably, the materials are metallic biocompatible. The material may be porous or non-porous, and the porous structural elements can be microporous, microstructure, or nanoporous.

The sintering process can be affected thermally, a method of which is described herein. The morphology of the layer, e.g. sphere size, thickness and tortuosity can be adjusted at the point of manufacture to accommodate the need for different elution profiles as may be required by the medical application at hand. Within the same medical application, e.g. the treatment of coronary restenosis, different morphologies may be desired to accommodate different elution profiles for different therapeutic agents. Some of the loading methods allow deposition of dilute or extremely dense crystalline forms of therapeutic agents within the structure thereby allowing a wide range of control over initial payloads. Such a stent can include the following: a supporting structure configured and dimensioned to be used within a body of an animal; a matrix body disposed on and at least partially covering the supporting structure, said matrix body having a plurality of cavities; a therapeutically effective amount of an active agent disposed within at least a portion of the structure, said therapeutically effective amount of the active agent being capable of treating and/or preventing a disease; and an elution rate controlling matrix disposed on at least one surface of the body so as to contain the active agent within said at least a portion of the voids, said matrix material that controls an elution rate of the active agent from the cavities. In some embodiments, the matrix void volume can be adjusted from 10-80% of the total matrix volume; defined below in greater detail. Further, the volume can be adjusted to accommodate the therapeutic formulation. For example, in a preferred embodiment the therapeutic is rapamycin at a concentration of 5-10 ug per millimeter of length of the stent.

In another embodiment of the present invention, the method of manufacturing can include the following: fabricating a supporting structure, which can include shaping the supporting structure into the stent and fabricating a matrix structure onto at least a portion of the supporting structure. In this embodiment the matrix can be coated selectively on either the abluminal or luminal surface or both. Then the stent can be cut from the support structure with non-matrix coated sides. A therapeutic agent and or elution controlling polymer can be introduced at anytime after the matrix is formed.

Various types of elution controlling polymer may be comprised of phosphorylcholines, phosphorylcholine linked macromolecules, polyolefins, poly(meth)acrylates, polyurethanes, polyesters, polyanhydrides, polyphosphazenes, polyacrylates, acrylic polymers, poly(lactide-coglycolides) (PLGA), polylactic acids (PLA), poly(hydroxybutyrates), poly(hydroxybutyrate-co-valerates), polydioxanones (PDO), polyorthoesters, polyglycolic acids (PGA), polycaprolactones (PCL), poly(glycolic acid-co-trimethylene carbonates), polyphosphoesters, polyphosphoester urethanes, poly(amino acids), cyanoacrylates, poly(trimethylene carbonate), poly(iminocarbonate), polyalkylene oxalates, polyiminocarbonates, aliphatic polycarbonates, fibrins, fibrinogens, celluloses, starches, collagens, polycarbonate urethanes, polyisoprenes, polyisobutylenes, polybutadienes, polyethylenes, plasticized polyethylene terephthalates, polyethylene terepthalates, polymethylmethacrylates, ethylene ethylacrylates, polyethyl hexylacrylates, plasticized ethylene vinylacetates, polyvinyl acetates, ethylene vinyl acetates, ethylene vinyl alcohols, polyvinyl alcohols, cross-linked polyvinyl alcohols, cross-linked polyvinyl butyrates, polyvinylbutyrates, polybutylmethacrylates, polyvinyl chlorides, ethylene vinylchloride copolymers, silicones, polysiloxanes, substituted polysiloxanes, polyethylene oxides, polyethylene glycols (PEG), polybutylene terepthalate-co-PEG, PCL-co-PEG, PLA-co-PEG, polyvinyl acetals, polyvinyl acetates, polyamides, polyvinyl pyrrolidones, polyacrylamides, polyvinyl esters, copolymers thereof, polymer derivatives thereof, or combinations thereof.

Various types of therapeutic agents are known in the art and commonly comprised of at least one of analgesics, antipyretics, antiasthamatics, antibiotics, antidepressants, antidiabetics, antifungal agents, antihypertensive agents, anti-inflammatories including non-steroidal and steroidal, antineoplastics, antianxiety agents, immunosuppressive agents, antimigraine agents, sedatives, hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines, anti-restenosis agents, antipruritics, agents useful for calcium regulation, antibacterial agents, antiviral agents, antimicrobials, anti-infectives, bronchodilators, steroidal compounds and hormones, or combinations thereof. Preferably, the active agent comprises at least one of rapamycin, rapamycin analog, Biolimus A9, zotarolimus, sirolimus, everolimus, dexamethasone, prednisone, hydrocortisone, estradiol, acetaminophen, ibuprofen, naproxen, sulidac, heparin, taxol, paclitaxel, and combinations thereof.

Typically the manufacturing process requires that the metal, metallic microspheres, to be applied as a matrix that is premixed with a sacrificial polymer such as polyurethane. The mixture can be applied to the substrate surface via spraying, direct coating applications, dipping, rolling or other know methods in the art. The coating thickness is typically between 10-30 microns because the coatings may be stacked to thicken the matrix. For example, the first coating will be heat treated to bond the metal particles and evaporate the sacrificial polymer. The heating environment should be non-reactive for cobalt chromium, and a preferred method utilizes an inert gas such as Argon or Helium. Different metals may require a different sintering environment to promote proper matrix pore size. After the polymer is completely removed, the substrate and matrix must be cooled. In the preferred embodiment the cooling would take place in an inert gas to reduce or eliminate the possibility of oxidizing the cobalt chromium. Subsequent, coatings of the premix of metal particles and polymer can be applied as described above and the substrate can be heat treated again. In one embodiment for example, the matrix after three coating steps was 50 microns thick. However, as described previously the matrix thickness can be adjusted, usually to about 20-150 microns.

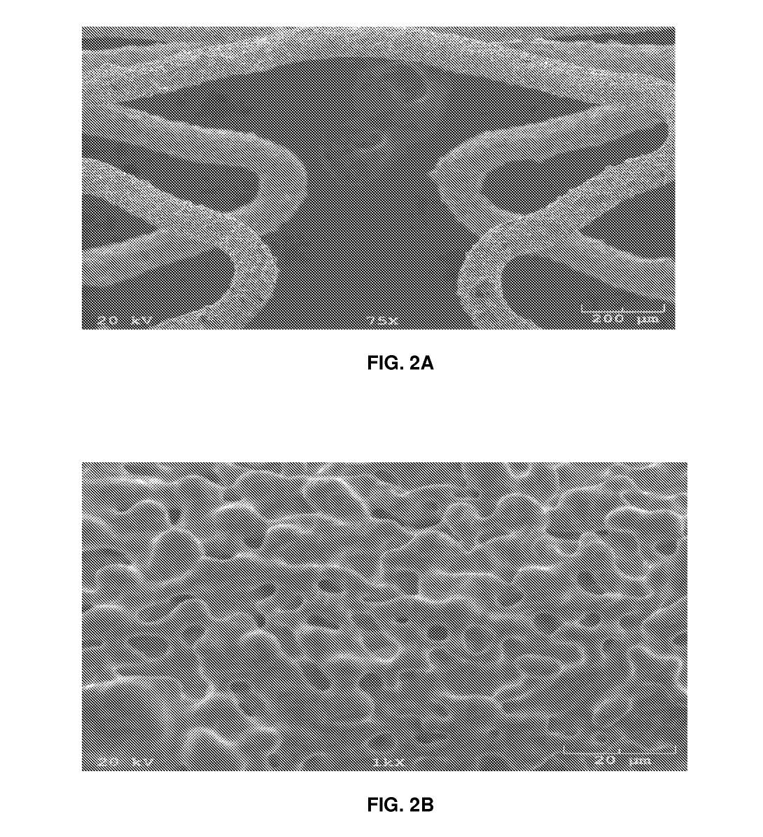

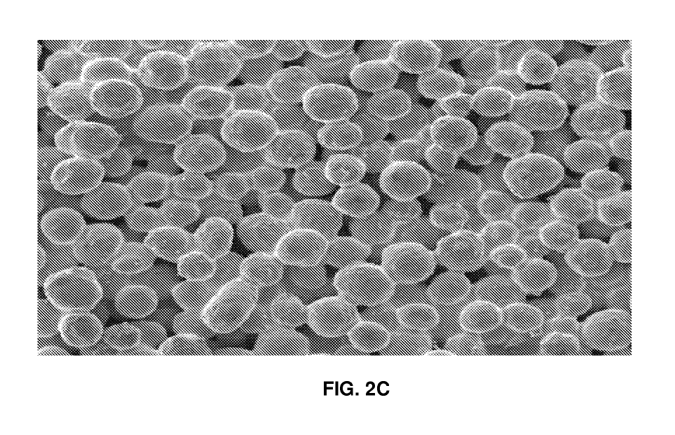

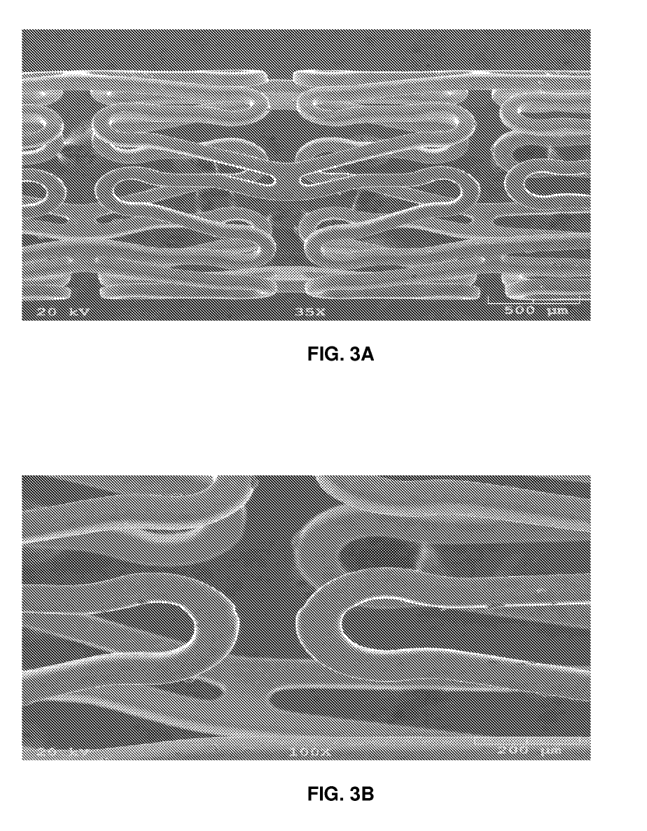

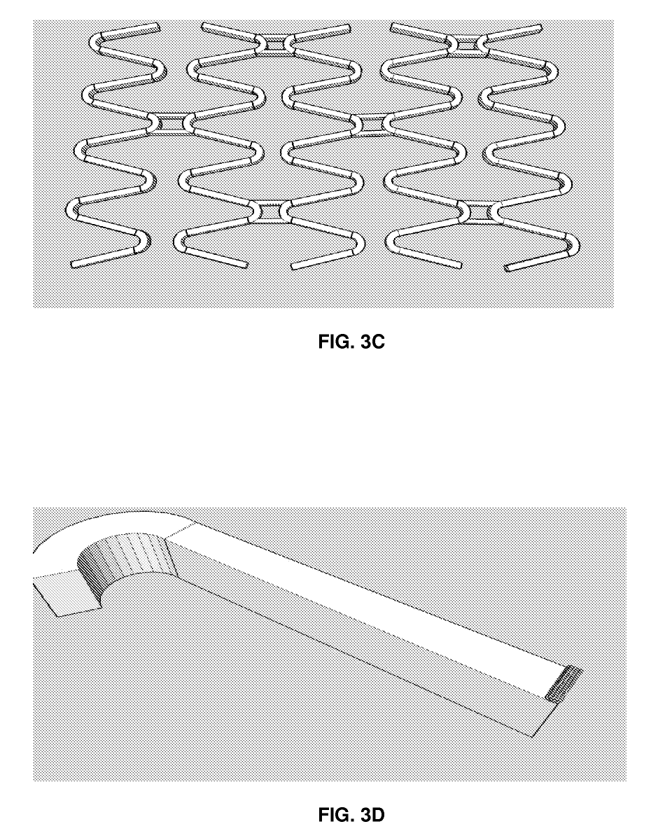

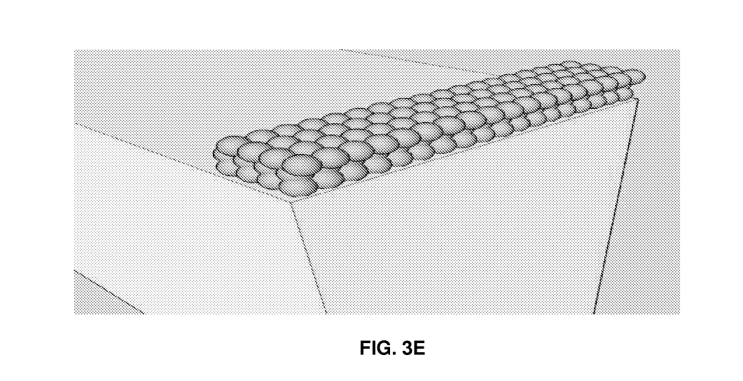

As shown in FIG. 2B, the elevated temperature and long exposure time made the beads melt thus the points of contact between them has increased. In an embodiment of the present invention it is preferable that cobalt chromium beads of size between 200 to 325 mesh are exposed to temperature at approximately 1246 degree Celsius for at least 30 minutes under vacuum. Of critical import to the success of the present invention is the void size distribution which will determine the total volume of void in the matrix.

The matrix layer can be characterized by a void fraction, defined as the fraction of open volume occupied by the voids. Matrix layers with higher void fractions can deliver larger amounts of therapeutic agents for the same thickness. Preferably, the void fraction is between about 10% to about 80%. In some embodiments, the void fraction is preferably within the range of about 20% to about 60%. The void fraction may also vary across different portions of the matrix layer. These features of the matrix layer may be measured using any of a variety of pore analysis products, such as those manufactured by Thortex, Inc. (Portland, Oreg.)

In a preferred embodiment the lumen side of the stent can be masked with aluminum foil or other suitable mask and the outer surfaces sprayed with a glue, then a powdered matrix of metallic microspheres and sacrificial binder, such as polyurethane, is dusted over the glue to adhere to the stents outer surfaces. The stent can be baked at a relatively low temperature between one-hundred to two-hundred degrees centigrade for one to five minutes to set the metallic microspheres so that a second spray coating of glue may be applied and another layer of the powdered matrix with metallic microspheres and sacrificial binder may be applied. The stent is then baked at a relatively low temperature of two-hundred degrees centigrade to set the microspheres against the first layer of microspheres. The process can be repeated to attain the level of thickness desired. A preferred sintering temperature profile includes a baking temperature of 1246 degrees Celsius for about one to ten hours in hydrogen filled vacuum pressured oven. The stent is the cooled to room temperature so that it can be coated. The masking of the lumen surface can be extended to cover portions of the sides of the struts. For example, several layers of aluminum foil or polymer wrap can be placed around a mandrel and the stent can be slightly crimped to push the masking material to contact the lumen surface of the stent and up through the struts. It is possible to mask up to one-hundred percent of the strut surfaces. In an alternative embodiment, the lumen surface is not masked at all, and the entire surface of the stent is coated with the glue and powdered matrix of metallic microspheres and sacrificial binder to create the porous surface.

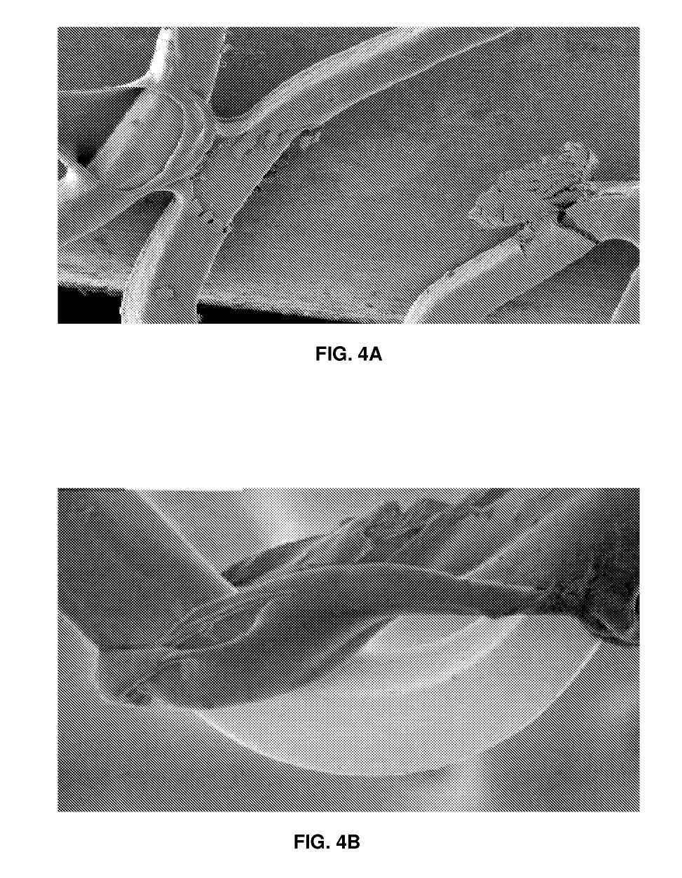

The materials typically used as carriers in coatings to drug eluting stent are polymeric materials such as poly(ethylene glycol)/poly(L-lactic acid) (PLGA). As described earlier, they have limitations related to coating adhesion, mechanical properties, and material biocompatibility. The structural integrity of existing coatings may be compromised during the use of the device. For example, radial expansion of a coronary stent may substantially disrupt the polymeric coating during deformation of the stent structure. FIG. 4A shows crack in the polymeric coating of a stent following balloon expansion. Polymeric coatings may also exhibit poor adhesion to a device even before expansion. FIG. 4B illustrates a separation of the polymeric coating from the stent structure after removal from its package. In both cases, there were no unusual circumstances which would predispose the polymeric coatings to crack or separate.

As implied in the term “drug-eluting stent” the compound and its pharmacologic properties hold a major key for the safety and efficacy of a DES. In contrast to release kinetics and polymer issues, there is considerable information available on how these drugs are different in terms of their anti-restenotic properties.

Among the typical examples of these agents that can be used individually or in different combinations in separate microsphere matrix are those discussed below as follows: the scope of our discussion is limited to two drugs; Paclitaxel and Rapamycin since the majority of other commercially approved drug are Rapamycin analogues for example, Everolimus (Guidant: Abbot Vascular, Zotralimus (Abbot), ABT578 (Medtronic), and many more. Additionally, release kinetics may be important for both efficacy and safety of a DES platform. Importantly, the presence and type of polymeric coating may also influence the rate of in-stent restenosis and stent thrombosis because polymers can be associated with ongoing vascular inflammation and delayed vascular healing. Paclitaxel is an antineoplastic compound which is used clinically in commercially available drug-eluting stents. Paxlitaxel can also be used as an anti-inflammatory agent with an exceptionally narrow therapeutic window beyond which it can be cytotoxic. Accordingly, the present invention provides for the use of paclitaxel in different fractions of the microsphere matrix in the capacity of an antineoplastic agent in combination with other drugs known for their anti-inflammatory activities (e.g., naproxen) and/or being immunosuppressant (e.g., rapamycin). Rapamycin is clinically used in commercially available drug-eluting stents. Rapamycin is also used as an immunosuppressant having a wide therapeutic window. However, its use in drug-eluting stents may not provide the optimum pharmacokinetics when released from a non-uniform coating.

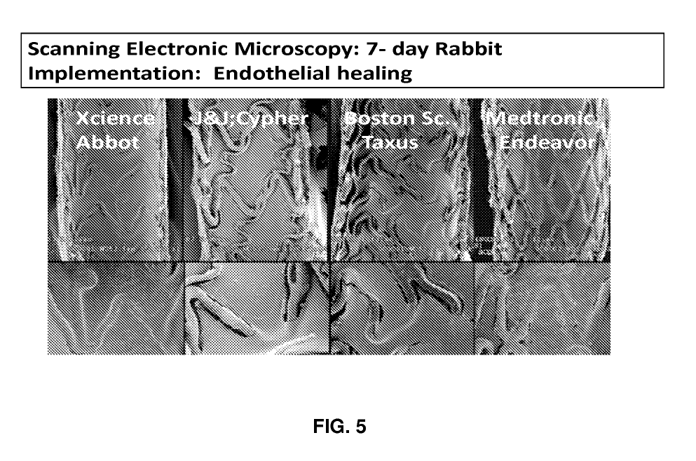

The effectiveness of both Rapamycin and Paclitaxel coated stents is dependent not only on the total delivered drug amount but also on release kinetics. For polymer coated rapamycin eluting stents, the results of four-year follow-up show that the slow-release rapamycin-coated stent, which is available as Cypher and maintains drug release for up to 60 days, has a more favorable outcome than a similar rapamycin eluting stent that releases its total dose within 7 days FIG. 5. With the Taxus stent there was no significant difference found between slow and medium release rates of paclitaxel eluting stents in a prospective human trial. In contrast, the polymer-free Supra-G stent (Cook, Bloomington, Ind.) showed a more favorable result in terms of restenosis for patients that received higher stent based paclitaxel dosages (3.1 μg/mm² vs. 1.3 μg/mm²). Taken together, recent findings from human trials suggest that the effectiveness of both paclitaxel and rapamycin may depend on total drug dosage as well as release kinetics. However, the optimal release kinetic may depend on lesion, patient characteristics, the stent matrix platform, and the therapeutic agent as well as the presence or absence of a polymer.

Accordingly, the present invention provides for the use of rapamycin or analogues at two drug loadings in different fractions of the microsphere matrix. For example, one layer may provide an initial burst and another layer may provide a prolonged, sustained release of the drug at lower concentrations. The present invention also may provide for use of rapamycin or analogues in combination with at least one additional bioactive agent, with different pharmacological activity. Typical examples of these other agents include endothelial cell growth promoters (e.g., vascular endothelial growth factor or its polypeptide functional analog), smooth muscle growth inhibitors, and antibiotics.

There are limited data regarding the effect of rapamaycin and paclitaxel on endothelial re-growth when polymer is present FIG. 5. It seems that both compounds retard endothelial regeneration, thus negatively affecting the restoration of its morphologic and functional integrity. This may facilitate, in some cases, the development of late stent thrombosis.

### Experiment #1

Seven 3.0 millimeter×14.3 millimeter cobalt chromium stents were processed as described above with a mask covering the lumen of the stent and having three coats to the abluminal surface and sides of the struts. The stents were baked under hydrogen vacuum for six hours and allowed to cool. The stents were then ultrasonic cleaned in acetone and allowed to dry. The stents were plasma cleaned prior to application of the drug formulation. Plasma cleaning involves the removal of impurities and contaminants from surfaces through the use of an energetic gaseous species such as argon and oxygen, as well as mixtures such as air and hydrogen/nitrogen are used. The plasma is created by using high radio-frequency to ionize a low pressure gas (usually 13.56 Mhz). The pressures of the gaseous species are typically below 1 Torr. The energetic, ionic species react with species on the surface of the stent, often producing gaseous products which can be removed by a vacuum system. The energetic species also clean the surface by collision with the surface, knocking off species from the surface. Prolonged or higher power plasma cleaning etches the surface, going beyond the cleaning phase. In this experiment all stents were treated for five minutes with a vacuum pressure set at 200 mTorr. The stents were weighed prior to coating with the results in Table 1, all weights are in micrograms.

TABLE 1

| | | Weight | Weight | Weight | | |
|---|---|---|---|---|---|---|
| | Stent # | 1 | 2 | 3 | Average | STD |
| Lumen | 1 | 17450 | 17448 | 17446 | 17448 | 2.00 |
| Masked | 2 | 16380 | 16392 | 16385 | 16386 | 6.03 |
| | 3 | 17244 | 17249 | 17242 | 17245 | 3.61 |
| | 4 | 16348 | 16352 | 16345 | 16348 | 3.51 |
| | 5 | 16674 | 16681 | 16679 | 16678 | 3.61 |
| | 6 | 16291 | 16293 | 16289 | 16291 | 2.00 |
| | 7 | 16012 | 16016 | 16020 | 16016 | 4.00 |

Then the stents were coated with rapamycin solution at a ratio of 1 gram rapamycin to 5 grams acetone and weighed. The results are in Table 2, all weights are in micrograms.

TABLE 2

| | Weight | Weight | Measured |
|---|---|---|---|
| Stent # | Before | After | Dose |
| 1 | 17448 | 17628 | 180 |
| 2 | 16386 | 16607 | 221 |
| 3 | 17245 | 17461 | 216 |
| 4 | 16348 | 16588 | 240 |
| 5 | 16678 | 16942 | 264 |
| 6 | 16291 | 16513 | 222 |
| 7 | 16016 | 16238 | 222 |

The average dose was 224 micrograms per stent. Stents were mounted on balloon catheters, sterilized and packaged. Stents were implanted in pigs; however, the results were unavailable for reporting because the thirty day endpoint had not been reached.

### Experiment #2

Eight 3.0 millimeter×14.3 millimeter cobalt chromium stents were processed as described above without a mask covering the lumen of the stent and having all of the stent surfaces available for coating. The stents were baked under hydrogen vacuum for six hours and allowed to cool. The stents were then ultrasonic cleaned in acetone and allowed to dry. The stents were plasma cleaned prior to application of the drug formulation. Plasma cleaning involves the removal of impurities and contaminants from surfaces through the use of an energetic gaseous species such as argon and oxygen, as well as mixtures such as air and hydrogen/nitrogen are used. The plasma is created by using high radio-frequency to ionize a low pressure gas (usually 13.56 Mhz). The pressures of the gaseous species are typically below 1 Torr. The energetic, ionic species react with species on the surface of the stent, often producing gaseous products which can be removed by a vacuum system. The energetic species also clean the surface by collision with the surface, knocking off species from the surface. Prolonged or higher power plasma cleaning etches the surface, going beyond the cleaning phase. In this experiment all stents were treated for five minutes with a vacuum pressure set at 200 mTorr. The stents were weighed prior to coating with the results in Table 3, all weights are in micrograms.

TABLE 3

| | Stent # | Weight 1 | Weight 2 | Weight 3 | Average | STD |
|---|---|---|---|---|---|---|
| No Mask | 1A | 16410 | 16420 | 16418 | 16416 | 5.29 |
| Group 2 | 2A | 16058 | 16062 | 16054 | 16058 | 4.00 |
| | 3A | 16386 | 16372 | 16389 | 16382 | 9.07 |
| | 4A | 16446 | 16448 | 16452 | 16449 | 3.06 |
| | 5A | 16464 | 16468 | 16463 | 16465 | 2.65 |
| | 6A | 16282 | 16287 | 16293 | 16287 | 5.51 |
| | 7A | 16358 | 16359 | 16363 | 16360 | 2.65 |
| | 8A | 16684 | 16689 | 16681 | 16685 | 4.04 |

Then the stents were coated with rapamycin solution at a ratio of 1 gram rapamycin to 5 grams acetone and weighed. The results are in Table 2, all weights are in micrograms.

TABLE 4

| | | Weight | Weight | Measured |
|---|---|---|---|---|
| | Stent # | Before | After | Dose |
| No Mask | 1A | 16416 | 16626 | 210 |
| Group 2 | 2A | 16058 | 16316 | 258 |
| | 3A | 16382 | 16618 | 236 |
| | 4A | 16449 | 16705 | 256 |
| | 5A | 16465 | 16687 | 222 |
| | 6A | 16287 | 16523 | 236 |
| | 7A | 16360 | 16576 | 216 |
| | 8A | 16685 | 16949 | 264 |

The average dose was 237 micrograms per stent. Stents were mounted on balloon catheters, sterilized and packaged. Stents were implanted in pigs; however, the results were unavailable for reporting because the thirty day endpoint had not been reached.

It will be understood that various modifications can be made to the various embodiments of the present invention herein disclosed without departing from the spirit and scope thereof. For example, various matrices and delivery device are contemplated as well as various types of construction materials. Also, various modifications may be made in the configuration of the parts and their interaction. Therefore, the above description should not be construed as limiting the invention, but merely as an exemplification of preferred embodiments thereof. Those of skill in the art will envision other modifications within the scope and spirit of the present invention as defined by the claims appended hereto.

What is claimed is:

1. An intravascular drug eluting stent comprising: a metallic substrate comprising a metallic microsphere matrix to carry one or more therapeutic agents; wherein when the stent is implanted into a blood vessel, the therapeutic agent is released from the stent through a controlled release profile of the agent to reduce various vessel disorders such atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, urethra obstruction, tumor obstruction, and combinations thereof, wherein the microsphere matrix will allow the endothelium lining ingrowth.

2. An intravascular drug eluting stent of claim 1 which the stent itself will be made of a metallic material or an alloy such as, but not limited to, cobalt-chromium alloys (e.g., ELGILOY), stainless steel (316L), “MP35N,” “MP20N,” ELASTINITE (Nitinol), tantalum, tantalum-based alloys, nickel-titanium alloy, platinum, platinum-based alloys such as, e.g., platinum-iridium alloy, iridium, gold, magnesium, titanium, titanium-based alloys, zirconium-based alloys, or combinations thereof. “MP35N” and “MP20N” are trade names for alloys of cobalt, nickel, chromium and molybdenum available from Standard Press Steel Co. of Jenkintown, Pa. “MP35N” consists of 35% cobalt, 35% nickel, 20% chromium, and 10% molybdenum, “MP20N” consists of 50% cobalt, 20% nickel, 20% chromium, and 10% molybdenum.

3. An intravascular drug eluting stent of claim 1 which the substrate will be made of a metallic material or an alloy such as, but not limited to, cobalt-chromium alloys (e.g., ELGILOY), stainless steel (316L), “MP35N,” “MP20N,” ELASTINITE (Nitinol), tantalum, tantalum-based alloys, nickel-titanium alloy, platinum, platinum-based alloys such as, e.g., platinum-iridium alloy, iridium, gold, magnesium, titanium, titanium-based alloys, zirconium-based alloys, or combinations thereof. “MP35N” and “MP20N” are trade names for alloys of cobalt, nickel, chromium and molybdenum available from Standard Press Steel Co. of Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum

4. An intravascular drug eluting stent which the microsphere matrix will be made of a metallic material or an alloy such as, but not limited to, cobalt-chromium alloys (e.g., ELGILOY), stainless steel (316L), “MP35N,” “MP20N,” ELASTINITE (Nitinol), tantalum, tantalum-based alloys, nickel-titanium alloy, platinum, platinum-based alloys such as, e.g., platinum-iridium alloy, iridium, gold, magnesium, titanium, titanium-based alloys, zirconium-based alloys, or combinations thereof. “MP35N” and “MP20N” are trade names for alloys of cobalt, nickel, chromium and molybdenum available from Standard Press Steel Co. of Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum

5. An intravascular drug eluting stent of claim 1, wherein the microsphere matrix will be made by a sintering process.

6. An intravascular drug eluting stent of claim 5, wherein the sintering process controls factors such as size distribution of the voids, size gradient of the voids, thickness of the coating, tortuosity of a porous network in the coating, surface roughness factor of the pores, or adsorption or chemosorption potential of the agent on the surface inside or outside the pores, a topcoat, or combinations of these.

7. The drug eluting stent of claim 1, wherein the metallic coating has a volume fraction of pores ranging from about 0.01 to about 0.5 millimeters.

8. The Drug Eluting Stent of claim 1, which is a coronary stent.

9. A method of forming a drug eluting stent comprising a microsphere metallic or coating that comprises matrix having a bioactive agent loaded therein, comprising forming the microsphere, metallic matrix coating comprising micropores, and loading the bioactive agent into the matrix.

10. An intravascular drug eluting stent of claim 1, wherein the bioactive agent is selected from the group consisting of paclitaxel, docetaxel, estradiol, nitric oxide donors, super oxide dismutases, super oxide dismutases mimics, 4-amino-2,2,6,6-tetramethylpiperidine-1-oxyl(4-amino-TEMPO), tacrolimus, dexamethasone, rapamycin, rapamycin derivatives, 40-O-(2-hydroxy)ethyl-rapamycin-(everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), Biolimus A9, clobetasol, pimecrolimus, imatinib mesylate, midostaurin, prodrugs thereof, co-drugs thereof, and a combination thereof.

11. The method of claim 9, wherein the loading comprises providing a solution comprising the agent, exposing the metallic matrix to the solution, and allowing the bioactive agent to diffuse into the cavities.

12. An intravascular drug eluting stent of claim 10, wherein the bioactive agent elutes into vessel wall leaving a void in the metallic matrix.

13. An intravascular drug eluting stent of claim 12, wherein, the endothelium line cell growth into the microsphere matrix.
